(19) United States
(12) Patent Application Publication
(10) Pub. No.: US 2008/0161921 A1
(43) Pub. Date: Jul. 3, 2008

(54) Spinal Prosthesis Systems

(75) Inventors: Tommy Carls, Memphis, TN (US); Jeffrey Zhang, Collierville, TN (US); Randall Allard, Germantown, TN (US); Chris Hughes, Lakeland, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/618,351

(22) Filed: Dec. 29, 2006

(51) Int. Cl. A61F 2/44; A61B 17/58

(52) U.S. Cl. 623/17.11; 606/74

(57) ABSTRACT

Spinal prosthetic devices having selectable and/or changeable centers of rotation are disclosed. In one aspect, a spinal prosthesis kit includes a first component, a second component, and a plurality of inserts. The first component has a first articulating surface that includes a first articulating structure. The second component has a second articulating surface with a recessed opening adapted to receive one of the plurality of inserts. Each of the plurality of inserts includes a second articulating structure adapted to moveably mate with the first articulating structure. The second articulating structure is positioned in a different location on each of the plurality of inserts. In some embodiments, the articulating structures are moveably mating projections and recesses. In another aspect, a revisable prosthetic device having a first component, a second component, and an insert is provided.

### TECHNICAL FIELD

Embodiments of the present disclosure relate generally to devices and methods for spinal surgery, and more particularly in some embodiments, to motion-preserving prosthetic devices having a selectable or changeable center of rotation.

### BACKGROUND

Although existing devices and methods have been generally adequate for their intended purposes, they have not been entirely satisfactory in all respects.

### SUMMARY

In one embodiment, a prosthetic kit is provided.

In a second embodiment, a prosthetic device is provided.

In another embodiment, a spinal prosthesis kit is provided. The kit includes a first component, a second component, and a plurality of inserts. The first component has a first engagement surface and an opposing first articulating surface. The first articulating surface includes a recess having a shape substantially similar to a portion of a sphere with a first radius of curvature. The second component has a second engagement surface and an opposing second articulating surface. The second articulating surface includes a recessed opening adapted to receive one of the plurality of inserts. The plurality of inserts for insertion into the recessed opening of the second component each include a projection having a shape substantially similar to a portion of a sphere with a second radius of curvature. The second radius of curvature is substantially equal to or less than the first radius of curvature. The projection of the insert is adapted to moveably mate with the recess of the first component. The projection is positioned in a different location on each of the plurality of inserts.

In another embodiment, a revisable prosthetic device for disposition within a space between a pair of vertebrae is provided. The device comprises a first component, a second component, and an insert. The first component includes a first engagement surface for engaging the first vertebra and an opposing first articulating surface. The first articulating surface includes a first articulating structure thereon. The second component includes a second engagement surface for engaging the second vertebra and an opposing second articulating surface. The second articulating surface includes a slot adapted to receive the insert. The insert includes a second articulating structure thereon. The second articulating structure is shaped to moveably mate with the first articulating structure of the first component.

In another embodiment, a spinal prosthesis kit for disposition within a space between a pair of vertebrae is provided. The kit comprises a first component, a second component, and a plurality of inserts. The first component includes a first engagement surface for engaging the first vertebra and an opposing first articulating surface. The first articulating surface includes a first articulating structure thereon. The second component includes a second engagement surface for engaging the second vertebra and an opposing second articulating surface. The second articulating surface includes a slot adapted to receive one of the plurality of inserts. Each of the plurality of inserts for insertion into the slot of the second component includes a second articulating structure thereon. The second articulating structure is shaped to moveably mate with the first articulating structure of the first component. The second articulating structure is located in a different position for each of the plurality of inserts.

Additional and alternative features, advantages, uses, and embodiments are set forth in or will be apparent from the following description, drawings, and claims.

### BRIEF DESCRIPTION OF THE DRAWINGS

FIG. 1 is a diagrammatic lateral view of a portion of a spondylosed vertebral column.

FIG. 2 is a diagrammatic lateral view of a pair of vertebrae.

FIG. 3 is a diagrammatic front view of a prosthetic device that embodies aspects of the present disclosure. (central center of rotation)

FIG. 4 is a side view of the prosthetic device of FIG. 2.

FIG. 5 is a top view of a portion of the prosthetic device of FIG. 2.

FIG. 6 is a top view of a portion of a prosthetic device similar to FIG. 5, but showing alternative embodiments in shadow.

FIG. 7 is a front view of a prosthetic device according to one of the alternative embodiments of FIG. 6.

FIG. 8 is a side view of the prosthetic device of FIG. 7.

FIG. 9 is a top view of a portion of a prosthetic device similar to FIGS. 5 and 6, but showing alternative embodiments in shadow.

FIG. 10 is a front view of a prosthetic device according to one of the alternative embodiments of FIG. 9.

FIG. 11 is a side view of the prosthetic device of FIG. 10.

FIG. 12 is a top view of a portion of a prosthetic device similar to FIGS. 5, 6 and 9, but showing alternative embodiments in shadow.

FIG. 13 is a front view of a prosthetic device according to one of the alternative embodiments of FIG. 12.

FIG. 14 is a side view of the prosthetic device of FIG. 13.

FIG. 15 is a diagrammatic front view of a prosthetic device similar to FIGS. 3, 7, 10, and 13, but showing an alternative embodiment.

FIG. 16 is a diagrammatic side view of a prosthetic device similar to FIGS. 4, 8, 11, and 14, but showing an alternative embodiment.

### DESCRIPTION

For the purposes of promoting an understanding of the principles of the invention, reference will now be made to the embodiments, or examples, illustrated in the drawings and specific language will be used to describe the embodiments. It will nevertheless be understood that no limitation of the scope of the invention is intended. Any alterations and further modifications of the described embodiments, and any further applications of the principles of the invention as described herein are contemplated as would normally occur to one skilled in the art to which the invention relates.

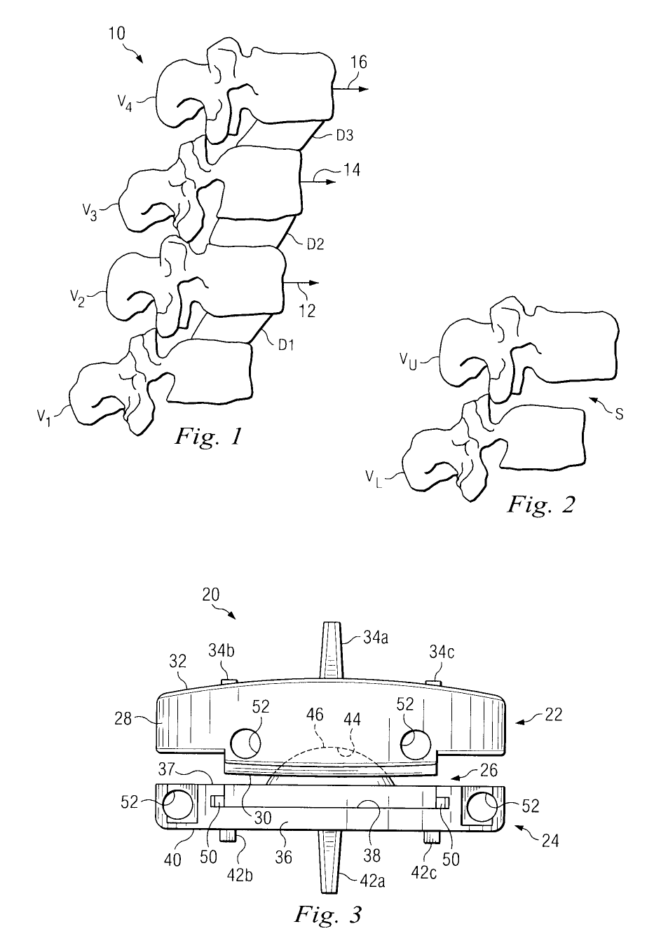

Referring to FIG. 1, shown therein is a lateral view of a portion of a spondylosed vertebral column 10, illustrated by a group of adjacent upper and lower vertebrae V1, V2, V3, V4 separated by intervertebral discs D1, D2, D3. As shown in the drawing, the vertebra V2 is dislocated from the vertebra V1 in a direction shown by arrow 12. Similarly, vertebra V3 is dislocated from the vertebra V2 in a direction shown by arrow 14 and vertebra V4 is dislocated from the vertebra V3 in a direction shown by arrow direction 16. In some patients it may be desirable to correct such misalignment and/or dislocation. Thus, in some embodiments the prosthetic devices described below are adapted to correct such misalignment and dislocation. In other embodiments, the prosthetic devices described below are adapted for use with such misalignment and dislocation, but are not adapted to correct the misalignment and dislocation.

Referring more specifically to FIG. 2, shown therein is a diagrammatic lateral view of a pair of spondylosed vertebrae, an upper vertebra V_{U} and a lower vertebra V_{L}. The vertebrae V_{U}, V_{L} may be located in any region of the spine, including the cervical, thoracic, lumbar, and sacral regions. In that regard, in some instances the lower vertebra V_{L} is the sacrum. In some spinal surgeries, some or all of the natural disc that would have been positioned between the two vertebrae V_{U}, V_{L} is removed via a discectomy or a similar surgical procedure, the details of which would be known to one of ordinary skill in the art. Removal of the diseased or degenerated natural intervertebral disc results in the formation of an intervertebral space S between the upper and lower vertebrae V_{U}, V_{L}. In some embodiments, the prosthetic devices described below are adapted for use and disposition within the intervertebral space S between the spondylosed vertebrae V_{U}, V_{L}. In some embodiments, the prosthetic devices described below are adapted for use and disposition between vertebrae that are not spondylosed. Thus, reference will be made to positioning the prosthetic devices between an upper vertebra V_{U} and a lower vertebra V_{L} and this is understood to include vertebrae of any anatomical orientation, including both spondylosed and non-spondylosed orientations.

For simplicity the prosthetic devices are described below as being adapted for use with an anterior surgical approach. However, in other embodiments the shape of the prosthetic devices, the positioning of the engagement structures thereon, and the insert pieces for defining the center of rotation are configured for other surgical approaches including, but not limited to posterior, lateral, oblique surgical approaches, and combinations thereof. Thus, the prosthetic devices and concepts described herein are adaptable for use in the surgical approach that will be most beneficial to the patient.

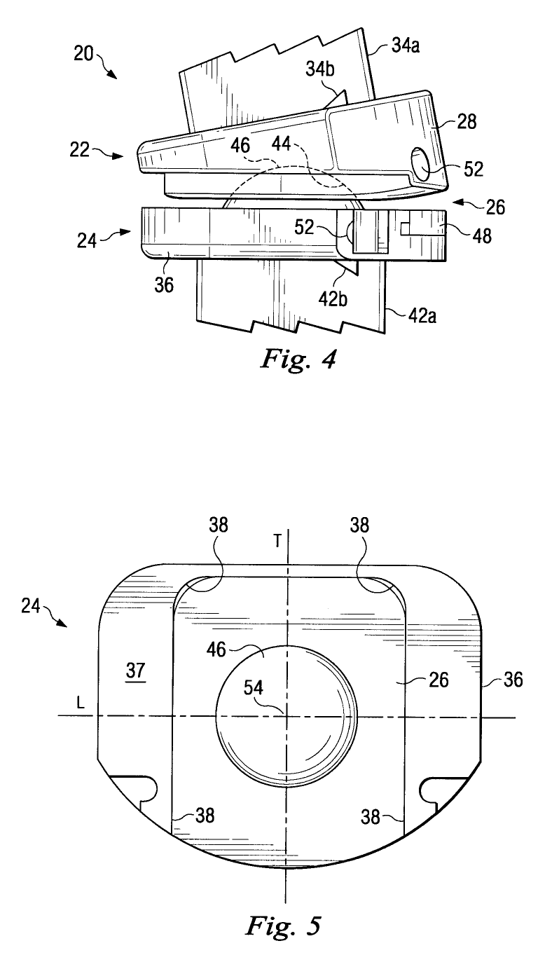

Referring to FIGS. 3-5, shown therein is a prosthetic device 20 according to one embodiment of the present disclosure. The prosthetic device 20 extends generally along a longitudinal axis L and includes an upper component 22, a lower component 24, and an articulating insert 26. The upper and lower components 22, 24 and the insert 26 cooperate to form the articulating device 20, which is sized and configured for disposition within an intervertebral space between a pair of vertebral bodies, such as the intervertebral space S between the adjacent vertebral bodies V_{U}, V_{L}. The upper component 22 comprises a support plate 28 having an articulating surface 30 and an opposing bearing surface 32. Engagement structures 34a, 34b, and 34c extend from the bearing surface 32. The engagement structures 34a, 34b, and 34c are adapted to facilitate secure engagement of the upper component 22 with the adjacent vertebra V_{U}.

The lower component 24 includes a support plate 36 having an articulating surface 37. The articulating surface 37 includes a recessed opening or slot 38. The slot 38 is adapted to receive and mate with the insert 26. The support plate also includes a bearing surface 40 opposite the articulating surface 37. Engagement structures 42a, 42b, and 42c extend from the bearing surface 40. The engagement structures 42a, 42b, and 42c are adapted to facilitate secure engagement of the lower component 24 with the adjacent vertebra V_{L}.

The prosthetic device 20 and, in particular, the interaction between the articulating surface 30 of the upper component 22 and the insert 26 provide relative pivotal, rotational, and/or translational movement between the adjacent vertebral bodies V_{U}, V_{L} to maintain and/or restore at least some motion similar to the normal bio-mechanical motion provided by a natural intervertebral disc. In some embodiments the interaction between the articulating surface 30 and the insert 26 comprises a ball-and-socket joint. In the current embodiment, the articulating surface 30 of the upper component 22 includes an a recess 44 adapted to pivotally mate with a projection 46 extending from a base plate 48 of the insert 26. Together the recess 44 and projection 46 form the motion-preserving ball-and-socket joint. In this manner, the upper component 22 and the insert 26 are configured to permit pivotal motion of the prosthetic device 20 and the adjacent vertebrae V_{U}, V_{L} about a number of axes, including lateral or side-to-side pivotal movement about the longitudinal axis L, anterior-posterior pivotal movement about a transverse axis T, rotational pivotal movement about a rotational axis R (perpendicular to both the longitudinal axis L and the transverse axis T), and combinations thereof.

In the current embodiment, the projection 46 is substantially convex and the recess 44 is substantially concave and sized to receive the projection. More particularly, in the current embodiment the projection 46 is substantially shaped as a portion of spherical ball and the recess 44 is substantially shaped as a portion of a spherical socket sized to receive the projection. In some embodiments the spherically shaped recess 44 has a radius of curvature substantially equal to or greater than the radius of curvature of the projection 46. The closer to equal the radii of curvature are, the more limited and/or defined the range of motion allowed by the prosthetic device 20 is.

It should be understood that in other embodiments the recess 44 and projection 46 have other shapes, such as, for example, cylindrical, elliptical, other arcuate configurations, and/or non-arcuate configurations. In general, the various embodiments of recesses and projections may be referred to as articulating structures. The recess 44 is shaped to mate with the projection 46 of the lower component 24 to provide at least some motion to the prosthetic device 20. Further, although the recess 44 is illustrated as having a generally smooth, uninterrupted surface, it should be understood that a surface depression, cavity, or groove may be defined along a portion of the recess to aid in clearing out matter, such as particulate debris, that is disposed between the abutting articulating surfaces the recess and the projection 46. In one such embodiment, the surface of the projection 46 defines a generally smooth, uninterrupted articular surface. Similarly, although the projection 46 is illustrated as having a generally smooth, uninterrupted surface, it should be understood that a surface depression, cavity, or groove may be defined along a portion of the projection to aid in clearing out matter, such as particulate debris, that is disposed between the abutting articulating surfaces of the recess 44 and the projection. In one such embodiment, the surface of the recess 44 defines a generally smooth, uninterrupted articular surface. In another embodiment, each of the recess 44 and the projection 46 define a surface depression to facilitate removal of particulate matter disposed between the abutting articulating surfaces.

In the current embodiment, the remaining portion of the articulating surface 30, the portion not defined by the recess 44, is substantially planar. However, in some embodiments the remaining portion of the articulating surface 30 is contoured to further facilitate the motion-preserving features of the prosthetic device 20 in some embodiments. For example, in some embodiments the remaining portion of the articulating surface 30 is angled or sloped as it extends from the edge of the recess 44 to the edges of the articulating surface 30 to allow for a greater range of motion than would be possible if the remaining portion of the articulating surface was planar. In other embodiments, the articulating surface 30 includes stop portions, is angled in one direction but not another direction, and/or is otherwise configured to facilitate and/or define the motion-preserving features, facilitate insertion, and/or facilitate the general use of the prosthetic device 20. Similarly, the remaining portion of the articulating surface of the insert 26, the portion not defined by the projection 46, may include similar features in various embodiments.

The slot 38 is adapted to receive and mate with the insert 26. In particular, the slot 38 is sized to receive the base plate 48 of the insert 26. In the current embodiment, the slot 38 is bounded by two sidewalls and a rear wall. In other embodiments, the slot 38 is not bounded by a rear wall. In some embodiments, the slot and/or the base plate include features to allow for selective engagement between the lower component 24 and the insert 26. For example, in one embodiment the lower component 24 includes a system for retaining the insert similar to the systems described in patent application Ser. No. 10/662,928 filed Sep. 15, 2003 and entitled Revisable Prosthetic Device, herein incorporate by reference in its entirety. Thus, in some embodiments the lower component 24 includes a retention member that is moveable between a first position for allowing the insert 26 to slide within the slot 38 and a second position for inhibiting the sliding of the insert within the slot. Further, in some embodiments the lower component includes an access hole for selectively moving the retention member between the first position and the second position. Therefore, in some embodiments the retention member is selectively engageable such that the insert 26 can later be removed. In some embodiments, the insert 26 is secured to the lower component 24 by a screw, a pin, glue, adhesive, or other revisable engagement mechanism.

In some instances, it may be desirable to replace the insert 26 with another insert during a revision surgery. Thus, in such instances the insert 26 and/or the lower component 24 include features to assist in the removal of the insert 26. For example, in the current embodiment the lower component 24 includes recesses 50 adjacent slot 38 to facilitate removal of the insert 26 after the insert has been inserted into the slot 38. In particular, the recesses 50 provides a surgical instrument (not shown) room to grasp the insert 26. In other embodiments, the insert 26 itself includes features to facilitate engagement with a surgical instrument to allow removal of the insert from the slot 38. As appreciated by one skilled in the art, any number of various structures on both the insert 26 and the lower component 24 may be used to facilitate removal of the insert 26 including but not limited to recesses, projections, threaded openings, threaded projections, and other structures.

In other embodiments the lower component 24 and/or the insert 26 includes a retention member or mechanism to permanently engage the insert with the lower component. For example, in one embodiment the lower component 24 and/or the plate 48 include a series of projections that allow one-way movement such that once the insert 26 is inserted into the slot 38, it is permanently engaged with lower component unless the projections are broken off. In some embodiments, the insert 26 is permanently affixed to the lower component through a permanent glue or other permanent adhesive. As recognized by one skilled in the art, the insert 26 may be permanently secured to the lower component in numerous other ways. In some embodiments the insert 26 is integrally formed with the lower component 24.

In the current embodiment, the support plates 28, 36 and the upper and lower components 22, 24 are sized and shaped to substantially correspond to the size and shape of the vertebral endplate of an adjacent vertebra V_{U}, V_{L}. In other embodiments, the support plates 28, 36 are sized and shaped to correspond to only a portion of the vertebral endplate of the adjacent vertebra. For example, in one alternative embodiment the support plates 28, 36 are sized and shaped for use in a bilateral procedure. In some embodiments, the upper support plate 28 and the lower support plate 36 have different sizes.

In some embodiments, the prosthetic device 20 includes one or more notches 52, or other types of structure, for receiving and engaging with a corresponding portion of a surgical instrument (not shown) to aid in the manipulation and insertion of the prosthetic device 20 within the intervertebral space between the adjacent vertebrae. The surgical instrument (not shown) is preferably configured to hold the upper and lower components 22, 24 at a predetermined orientation and spatial relationship relative to one another during the manipulation and insertion of the prosthetic device 20, and to release the upper and lower components once properly positioned between the adjacent vertebrae.

As described above, the upper component 22 and the insert 26 are configured to permit pivotal motion of the prosthetic device 20 and the adjacent vertebrae V_{U}, V_{L} about a number of axes, including lateral or side-to-side pivotal movement about the longitudinal axis L, anterior-posterior pivotal movement about the transverse axis T, rotational pivotal movement about the rotational axis R, and combinations thereof. In the current embodiment, this motion is defined about a center of rotation 54 located at the center point of the prosthetic device 20 at the intersection of the longitudinal axis L and the transverse axis T, as shown in FIG. 5. Thus, in the current embodiment the recess 44 is substantially centered about the intersection of the longitudinal axis L and the transverse axis T of the upper component 22 and the projection 46 is substantially centered about the intersection of the longitudinal axis L and the transverse axis T of the lower component 24. However, in some instances it is desirable to have the center of rotation offset with respect to the longitudinal axis L, the transverse axis T, or both the longitudinal and transverse axes. Thus, in some embodiments the center of rotation 50 is offset with respect to the longitudinal axis L and/or the transverse axis T by having the projection 46 offset along at least one of the axes, having the recess 44 offset along at least one of the axes, having the recess 38 for receiving the insert 26 offset along at least one of the axes, and/or having other structural orientations to offset the center of rotation from the center point of the prosthetic device 20. In some embodiments (e.g., where the prosthetic device allows for translational motion), the center of rotation is an area, not a single point. In such embodiments, the center of rotation area may be offset along the longitudinal axis L and/or the transverse axis T. In that regard, though reference will now be made to offset centers of rotation in the context of a single point, it is to be understood that the concepts apply equally to center of rotation areas as well.

Reference will now be made to alternative embodiments of the prosthetic device 20 according to the present disclosure. However, in many aspects the alternative embodiments are substantially similar to the prosthetic device 20 and/or its various alternative features described above. Therefore, for simplicity the same reference numerals used to describe the prosthetic device 20 above will be used to describe the alternative embodiments below. However, it is to be understood that the alternative embodiments may include additional features not explicitly described in relation to the prosthetic device 20.

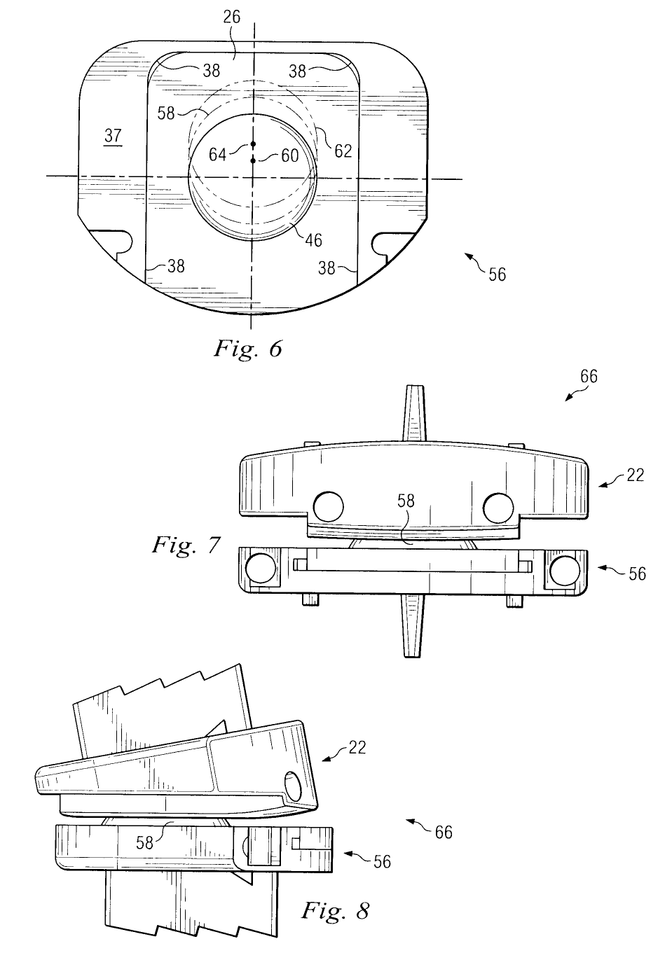

Referring now to FIG. 6, shown therein is a component 56 of a prosthetic device similar to the lower component 24 of the prosthetic device 20 above, but showing in shadow alternative locations for the projection 46 on the insert 26. In particular, FIG. 6 illustrates alternative locations for the projection 46 that are substantially aligned with the transverse axis T, but are offset with respect to the longitudinal axis L. For example, a projection 58 is shown in shadow with a center of rotation 60 that is offset with respect to the longitudinal axis L. Similarly, a projection 62 is shown in shadow with a center of rotation 64 that is offset with respect to the longitudinal axis L. The projections 58, 62 are merely exemplary locations for the offset projections. The offset projection 58, 62 of the component 56 may be offset at any distance from the longitudinal axis L. Further, the offset projection is offset in the opposite direction from the offset projections 58, 62 in some embodiments (e.g., towards the anterior portion of the device in the current embodiment if the projections 58, 62 are considered to be offset towards the posterior portion of the device).

Referring now to FIGS. 7 and 8, shown therein is a prosthetic device 66 similar to the prosthetic device 20 described above, but showing an alternative embodiment. In particular, the lower component 24 has been replaced with the component 56 having a projection with a center of rotation offset with respect to the longitudinal axis L. As shown in FIG. 7, the device 66 remains substantially aligned when viewed from the front. However, as shown in FIG. 8, when viewed laterally the upper component 22 and the lower component 56 are offset due to the projection with an offset center of rotation. As shown, in the current embodiment the recess of the upper component 22 that moveably engages the projection of the lower component is substantially centered with respect to longitudinal and transverse axes of the upper component. In other embodiments, the recess of the upper component 22 is offset with respect longitudinal and/or transverse axes of the upper component. For example, in one particular embodiment the recess of the upper component is offset with respect to the longitudinal axis of the of the upper component to the same degree the projection of the component 56 is offset with respect to the longitudinal axis L such that the components are substantially aligned, but the center of rotation offset from the center of the device.

Referring now to FIG. 6, shown therein is a component 56 of a prosthetic device similar to the lower component 24 of the prosthetic device 20 above, but showing in shadow alternative locations for the projection 46 on the insert 26. In particular, FIG. 6 illustrates alternative locations for the projection 46 that are substantially aligned with the transverse axis T, but are offset with respect to the longitudinal axis L. For example, a projection 58 is shown in shadow with a center of rotation 60 that is offset with respect to the longitudinal axis L. Similarly, a projection 62 is shown in shadow with a center of rotation 64 that is offset with respect to the longitudinal axis L. The projections 58, 62 are merely exemplary locations for the offset projections. The offset projection 58, 62 of the component 56 may be offset at any distance from the longitudinal axis L. Further, in some embodiments the offset projection is offset in the opposite direction from the offset projections 58, 62 (e.g., towards the anterior portion of the device in the current embodiment if the projections 58, 62 are considered to be offset towards the posterior portion of the device).

Referring now to FIGS. 7 and 8, shown therein is a prosthetic device 66 similar to the prosthetic device 20 described above, but showing an alternative embodiment. In particular, the lower component 24 has been replaced with the component 56 having the projection 58 with the center of rotation 60 that is offset with respect to the longitudinal axis L of the component. As shown in FIG. 7, the device 66 remains substantially aligned when viewed from the front. However, as shown in FIG. 8, when viewed laterally the upper component 22 and the lower component 56 are offset due to center of the projection 58 being positioned off of the longitudinal axis L. In particular, the projection 58 is closer to the left side or posterior portion of the device 66 as viewed in FIG. 8.

As shown, in the current embodiment the recess of the upper component 22 that moveably engages the projection 58 of the component 56 is substantially centered with respect to the longitudinal and transverse axes of the upper component. In other embodiments, the recess of the upper component 22 is offset with respect longitudinal and/or transverse axes of the upper component. For example, in one particular embodiment the recess of the upper component is offset with respect to the longitudinal axis of the of the upper component to the same degree the projection of the component 56 is offset with respect to the longitudinal axis L such that the components are substantially aligned, but the center of rotation is offset from the center of the device.

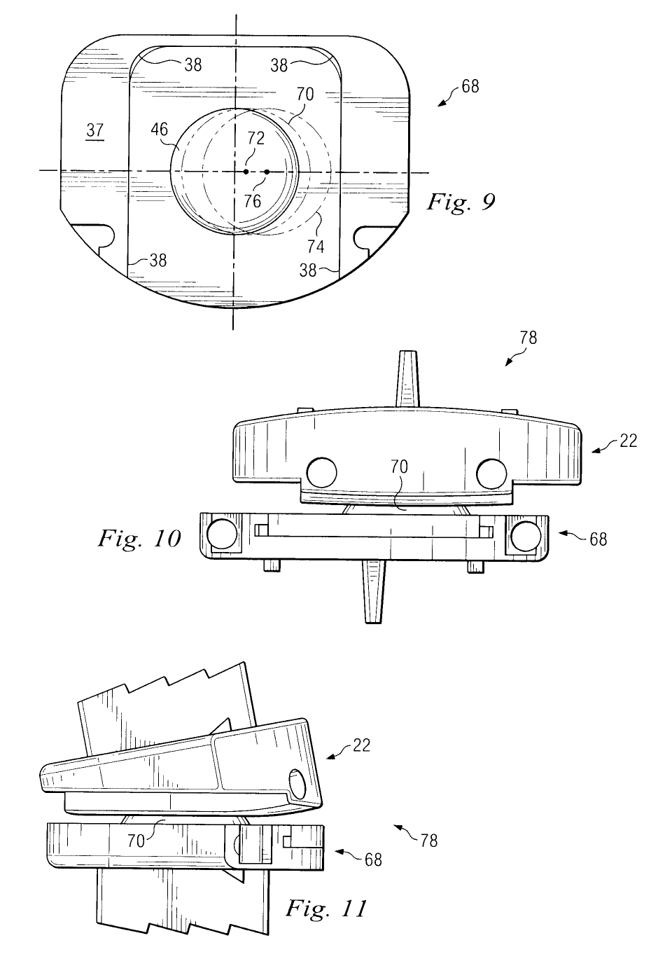

Referring now to FIG. 9, shown therein is a component 68 of a prosthetic device similar to the components 24 and 56 of the prosthetic devices above, but showing in shadow alternative locations for the projection on the insert 26. In particular, FIG. 9 illustrates alternative locations for the projection that are substantially aligned with the longitudinal axis L, but are offset with respect to the transverse axis T. For example, a projection 70 is shown in shadow with a center of rotation 72 that is offset with respect to the longitudinal axis L. Similarly, a projection 74 is shown in shadow with a center of rotation 76 that is offset with respect to the longitudinal axis L. The projections 70, 74 are merely exemplary locations for the offset projections. The offset projection 70, 74 of the component 68 may be offset at any distance from the longitudinal axis L. Further, in some embodiments the offset projection is offset in the opposite direction from the offset projections 70, 74 (e.g., towards the left lateral portion of the device in the current embodiment if the projections 58, 62 are considered to be offset towards the right lateral portion of the device).

Referring now to FIGS. 10 and 11, shown therein is a prosthetic device 78 similar to the prosthetic devices described above, but showing an alternative embodiment. In particular, the lower component has been replaced with the component 68 having the projection 70 with the center of rotation 72 that is offset with respect to the longitudinal axis L of the component. As shown in FIG. 10, when viewed from the front the upper component 22 and the lower component 68 are offset due to center of the projection 70 being positioned off of the transverse axis T. In particular, the projection 70 is closer to the right side of the device 78 as viewed in FIG. 10. However, as shown in FIG. 8, the device 78 remains substantially aligned when viewed from the side.

As shown, in the current embodiment the recess of the upper component 22 that moveably engages the projection 70 of the component 68 is substantially centered with respect to the longitudinal and transverse axes of the upper component. In other embodiments, the recess of the upper component 22 is offset with respect longitudinal and/or transverse axes of the upper component. For example, in one particular embodiment the recess of the upper component is offset with respect to the transverse axis of the of the upper component to the same degree the projection 70 of the component 68 is offset with respect to the transverse axis T such that the components are substantially aligned, but the center of rotation is offset from the center of the device.

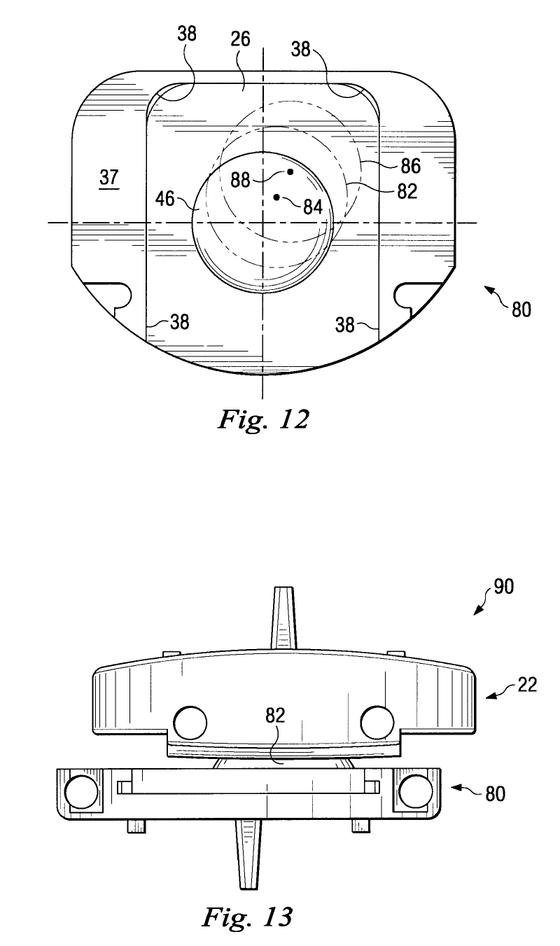

Referring now to FIG. 12, shown therein is a component 80 of a prosthetic device similar to the components 24, 56, and 68 of the prosthetic devices above, but showing in shadow alternative locations for the projection on the insert 26. In particular, FIG. 12 illustrates alternative locations for the projection that are offset with respect to both the longitudinal axis L and the transverse axis T of the component 80. For example, a projection 82 is shown in shadow with a center of rotation 84 that is offset with respect to the longitudinal axis L and the transverse axis T. Similarly, a projection 86 is shown in shadow with a center of rotation 88 that is offset with respect to the longitudinal axis L and the transverse axis T. The projections 82, 86 are merely exemplary locations for the offset projections. The offset projection 82, 86 of the component 80 may be offset at any distance from the longitudinal axis L and offset at any distance from the transverse axis T. Thus, according to the present embodiment the projection of the component 80 may be positioned anywhere on the insert 26 that is offset from both the longitudinal and transverse axes.

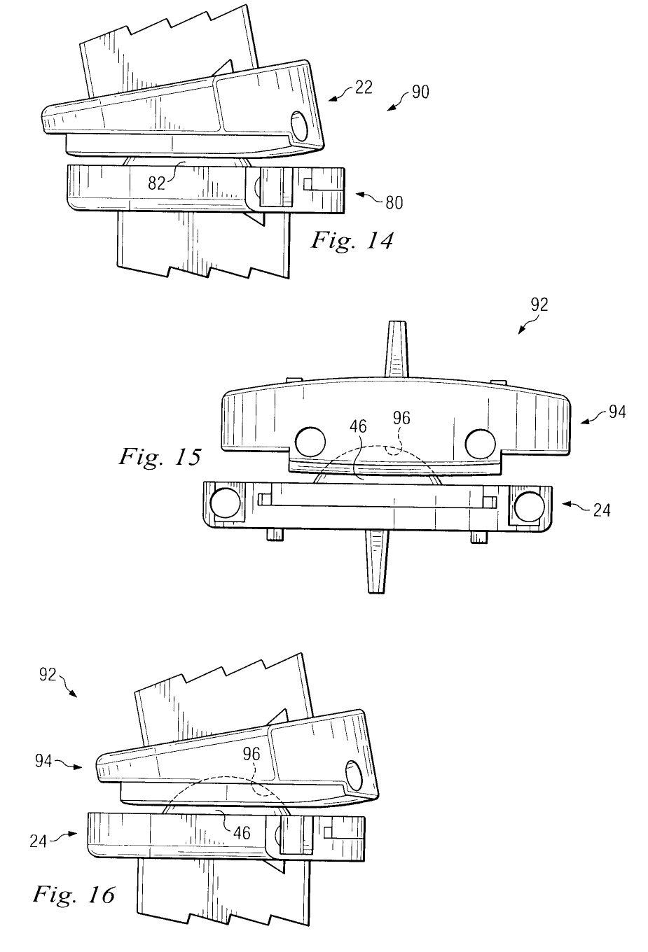

Referring now to FIGS. 13 and 14, shown therein is a prosthetic device 90 similar to the prosthetic devices described above, but showing an alternative embodiment. In particular, the lower component has been replaced with the component 80 having the projection 82 with the center of rotation 84 that is offset with respect to the longitudinal axis L of the component. As shown in FIG. 13, when viewed from the front the upper component 22 and the lower component 80 are offset due to center of the projection 82 being positioned off of the transverse axis T. In particular, the projection 82 is closer to the right side of the device 90 as viewed in FIG. 13. Similarly as shown in FIG. 14, when viewed laterally the upper component 22 and the lower component 80 are offset due to center of the projection 82 being positioned off of the longitudinal axis L. In particular, the projection 82 is closer to the left side or posterior portion of the device 66 as viewed in FIG. 14.

As shown, in the current embodiment the recess of the upper component 22 that moveably engages the projection 82 of the component 80 is substantially centered with respect to the longitudinal and transverse axes of the upper component. In other embodiments, the recess of the upper component 22 is offset with respect longitudinal and/or transverse axes of the upper component. For example, in one particular embodiment the recess of the upper component is offset with respect to the longitudinal and transverse axes of the of the upper component to the same degree the projection 82 of the component 80 is offset with respect to the longitudinal axis L and transverse axis T such that the components are substantially aligned, but the center of rotation is offset from the center of the device.

As mentioned above, in some embodiments the center of rotation is offset with respect to the longitudinal axis L and/or the transverse axis T not by having the projection offset, but rather by having the recess 44 of the upper component 22 offset along at least one of the axes, having the recess 38 for receiving the insert 26 of the lower component 24 offset along at least one of the axes, and/or having other structural orientations to offset the center of rotation from the center of the prosthetic device.

Referring now to FIGS. 15 and 16, shown therein is a prosthetic device 92 that is similar to the prosthetic devices described above, but showing an alternative embodiment. In particular, the upper component 22 has been replaced by a component 94 that has recess 96 with an offset center of rotation. As shown in FIG. 15, when viewed from the front the upper component 94 and the lower component 24 are offset due to center of the recess 96 being positioned off of the transverse axis of the upper component. In particular, the recess 96 is closer to the left side of the upper component as viewed in FIG. 15. Similarly as shown in FIG. 16, when viewed laterally the upper component 94 and the lower component 24 are offset due to center of the recess 96 being positioned off of the longitudinal axis of the upper component. In particular, the recess 96 is closer to the left side or posterior portion of the upper component 94 as viewed in FIG. 16. The offset of the recess 96 is exemplary only. It is understood that in other embodiments the recess 96 may be offset with respect to the longitudinal and transverse axes of the upper component to a greater or lesser extent, or be substantially aligned with the axes. Further, as shown the projection 46 of the lower component 24 is substantially centered about the intersection of the longitudinal axis L and the transverse axis T of the lower component. However, as disclosed above in other embodiments the projection 46 is offset with respect to the longitudinal axis L and/or the transverse axis T.

In at least one embodiment of the present disclosure, a prosthetic device kit is provided with a plurality of insert pieces. Each of the plurality of insert pieces having a projection with a different center of rotation. Thus, the surgeon may choose the insert with the center of rotation most appropriate for the particular patient. Similarly, in some embodiments the insert piece includes a recess instead of the projection. Thus, a plurality of insert pieces each having a recess with a different center of rotation may be provided in some embodiments. Further, though the lower component has been described as being configured to receive the insert, in other embodiments the upper component is configured to receive the insert. In some embodiments, both the upper and lower components are configured to receive an insert. Also, in some embodiments the upper component includes a projection and the lower component includes a recess adapted to moveably mate with the projection of the upper component. Further, the prosthetic device may be revisable such that an initial insert can later be replaced with an insert having a different center of rotation. In this manner, the prosthetic device can be modified to accommodate changes in the patient's spinal anatomy.

The components of the various prosthetic devices described above may be formed of any suitable biocompatible material including metals such as cobalt-chromium alloys, titanium alloys, nickel titanium alloys, or stainless steel alloys. Ceramic materials such as aluminum oxide or alumina, zirconium oxide or zirconia, compact of particulate diamond, or pyrolytic carbon may also be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); or cross-linked UHMWPE. Further, the components may each be formed of different materials, permitting metal on metal, metal on ceramic, metal on polymer, ceramic on ceramic, ceramic on polymer, or polymer on polymer constructions. Further, the articulating surfaces and the articulating portions described above may be treated to limit friction and resulting wear debris caused by rotational movement.

Further, the surfaces of the prosthetic devices that are positioned in direct contact with the bone structure, such as a vertebra, may be coated with a bone-growth promoting substance that would occur to one of ordinary skill in the art. Additionally, the surface of the prosthetic devices that are positioned in direct contact with vertebral bone may be roughened to further enhance bone in-growth. Such surface roughening may be accomplished by way of acid etching, knurling, application of a bead coating, or other methods of roughening that would occur to one of ordinary skill in the art. Further, surface roughening may be used in combination with bone-growth promoting substances. Also, the engagement structures described above may be replaced with any other suitable structures to facilitate engagement with the adjacent bone structure.

Other modifications of the present disclosure would be apparent to one skilled in the art. Accordingly, all such modifications and alternatives are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” and “right,” are for illustrative purposes only and can be varied within the scope of the disclosure. In the claims, means-plus-function clauses are intended to cover the structures described herein as performing the recited function and not only structural equivalents, but also equivalent structures.

1. A spinal prosthesis kit comprising:
a first component having a first engagement surface and an opposing first articulating surface, the first articulating surface including a recess having a shape substantially similar to a portion of a sphere with a first radius of curvature;
a second component having a second engagement surface and an opposing second articulating surface, the second articulating surface including a recessed opening adapted to receive an insert; and
a plurality of inserts for insertion into the recessed opening of the second component, each of the plurality of inserts including a projection having a shape substantially similar to a portion of a sphere with a second radius of curvature, the second radius of curvature being substantially equal to or less than the first radius of curvature, wherein the projection is adapted to moveably mate with the recess of the first component, and wherein the projection is positioned in a different location on each of the plurality of inserts.

2. The kit of claim 1 wherein the second component has a longitudinal axis extending substantially along its length and a transverse axis extending in a direction substantially perpendicular to the longitudinal axis, the transverse axis intersecting the longitudinal axis at a center point of the second component; and wherein the projection of at least one of the plurality of inserts is offset with respect to the center point of the second component after insertion of the insert into the recessed opening of the second component.

3. The kit of claim 2 wherein the projection of at least one of the plurality of inserts is offset with respect to the center point along the longitudinal axis of the second component after insertion of the insert into the recessed opening of the second component.

4. The kit of claim 2 wherein the projection of at least one of the plurality of inserts is offset with respect to the center point along the transverse axis of the second component after insertion of the insert into the recessed opening of the second component.

5. The kit of claim 2 wherein the projection of at least one of the plurality of inserts is substantially centered about the center point of the second component after insertion of the insert into the recessed opening of the second component.

6. The kit of claim 2 wherein the recess of the first component is substantially centered about a center point of the first component.

7. The kit of claim 2 wherein the recess of the first component is offset with respect to a center point of the first component.

8. The kit of claim 2 wherein the second component includes at least one recess adjacent the recessed opening, the at least one recess providing access to at least a portion of one side of the insert after insertion of the insert into the recessed opening.

9. The kit of claim 2 wherein the second component includes at least one retention member for retaining the insert within the recessed opening.

10. The kit of claim 9 wherein the at least one retention member is moveable between a first released position and a second retained position for selectively retaining the insert within the recessed opening.

11. A revisable prosthetic device for disposition within a space between a pair of vertebrae, the device comprising:
a first component having a first engagement surface for engaging the first vertebra and an opposing first articulating surface, the first articulating surface including a first articulating structure thereon, the first articulating structure substantially centered about a center point of the first component;
a second component having a second engagement surface for engaging the second vertebra and an opposing second articulating surface, the second articulating surface including a slot adapted to receive an insert; and
an insert for insertion into the slot of the second component, the insert including a second articulating structure thereon, the second articulating structure shaped to moveably mate with the first articulating structure of the first component, wherein the second articulating structure is offset with respect to a center point of the insert.

12. The device of claim 11 wherein the first articulating structure comprises a recess and the second articulating structure comprises a projection.

13. The device of claim 12 wherein the recess has a shape substantially similar to a portion of a sphere with a first radius of curvature, the projection has a shape substantially similar to a portion of a sphere with a second radius of curvature, the second radius of curvature being substantially equal to or less than the first radius of curvature.

14. The device of claim 13 wherein the recess is offset with respect to a center point of the first component.

15. The device of claim 13 wherein the projection is offset with respect to a center point of the second component after insertion of the insert into the slot.

16. The device of claim 15 wherein the first articulating structure comprises a projection and the second articulating structure comprises a recess.

17. The device of claim 16 wherein the recess is offset with respect to a center point of the second component after insertion of the insert into the slot.

18. The device of claim 17 wherein the insert is selectively removable from the slot after insertion.

19. The device of claim 11 wherein the second articulating structure is offset with respect to a center point of the second component after insertion of the insert into the slot.

20. The device of claim 19 wherein the first articulating structure is offset with respect to a center point of the first component.

21. A spinal prosthesis kit for disposition within a space between a pair of vertebrae, the kit comprising:
a first component having a first engagement surface for engaging the first vertebra and an opposing first articulating surface, the first articulating surface including a first articulating structure thereon;
a second component having a second engagement surface for engaging the second vertebra and an opposing second articulating surface, the second articulating surface including a slot adapted to receive an insert; and
a plurality of inserts for insertion into the slot of the second component, each of the plurality of inserts including a second articulating structure thereon, the second articulating structure shaped to moveably mate with the first articulating structure of the first component, wherein the second articulating structure is located in a different position for each of the plurality of inserts.

22. The kit of claim 21 wherein the first articulating structure comprises a recess and the second articulating structure comprises a projection.

23. The kit of claim 22 wherein the recess is substantially arcuate with a first radius of curvature and the projection is substantially arcuate with a second radius of curvature, the second radius of curvature being substantially equal to or less than the first radius of curvature.

24. The kit of claim 21 wherein the first articulating structure comprises a projection and the second articulating structure comprises a recess.
